(19) United States
(12) Patent Application Publication
(10) Pub. No.: US 2005/0090827 A1
(43) Pub. Date: Apr. 28, 2005

(54) Comprehensive tissue attachment system

(76) Inventor: Tewodros Gedebou, Los Angeles, CA (US)

Correspondence Address: MATTHEW A. NEWBOLES;STETINA BRUNDA GARRED & BRUCKER, Suite 250, 75 Enterprise, Aliso Viejo, CA, 92656, US

(21) Appl. No.: 10/695,271

(22) Filed: Oct. 28, 2003

(51) Int. Cl. A61B 17/58

(52) U.S. Cl. 606/72

(57) ABSTRACT

Bone anchors, methods for deploying anchors, and systems for attaching ligaments and tendons to bone are disclosed. The bone anchors of the present invention are operative to remain firmly positioned within a target site of bone and provide means for selectively adjusting the degree of tension to one or more sutures secured thereto. The bone anchors are further operative to be utilized to connect tendons and ligaments directly to bone, as well as may be selectively utilized to adjustably position an implant or other anatomical structure held thereby. There is further disclosed methods for deploying a bone anchor utilizing a blind procedure that is expeditious, accurate and substantially less traumatic than prior art bone deployment techniques.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

Surgical procedures requiring the attachment of anatomical structures, such as implants, sutures, grafts, slings, and the like to various points within the body are well-known. Typically, such anatomical structures are secured into position via a variety of anchoring mechanisms, known as surgical suture anchors or bone anchors, that typically provide a secure point of attachment to which an anatomical structure can be attached or otherwise supported. In this respect, such anchors are operative to attach a variety of anatomical structures and the like to bone and have found widespread applicability for a number of surgical procedures. In particular, bone anchors are extensively utilized in orthopedic and gynecologic procedures, as well as plastic and reconstructive surgery, where the same can be utilized to selectively shape and support specific regions of the body.

Generally, prior art bone anchors take one of two forms. The first configuration typically comprises a self-tapping bone screw made of a bio-compatible material, such as titanium, having an eyelet or some other like structure formed thereon to which a suture may be attached. FIG. 1 is illustrative of such prior art bone anchor. In the alternative prior art configuration (not shown), the device comprises an anchor member, which is typically defined by a generally conical shape, that is designed to be projected (i.e., “shot”) into bone at a desired position. Extending from the anchor member is a shaft or other type of attachment member such as an eyelet, which defines a structure to which a suture may be attached.

With respect to the surgical installation of such devices, bone-screw mechanisms must be screwed into position, typically by a battery powered screw driver, at a target site upon a particular bone. Anchor devices of the other aforementioned variety are forceably projected into position at a particular site within a bone typically via a spring-loaded delivery mechanism. Exemplary of such prior art surgical suture anchors include those disclosed in U.S. Pat. Nos. 5,370,662 and 5,443,482 to Stone, et al. and U.S. Pat. No. 4,738,255 to Gobel, et al., the teachings of which are expressly incorporated by reference.

Although such prior art bone anchor devices generally provide sufficient support to the various sutures and structures affixed thereto, such fixation devices suffer from several well-known draw backs. Perhaps most problematic with such devices is the inability to make any kind of adjustment once a suture is affixed thereto. In this regard, bone anchors typically are rigid structures that remain in a fixed, seated position, and offer no means by which a suture or other structure affixed thereto can be adjusted to impart a desired degree of tension and/or support. In such instances, the surgeon is relegated to having to cut and/or retie the suture line to such anchor until an optimal degree of tension and/or positioning is attained.

Such limitation further complicates post-operative procedures to the extent a given suture is sub-optimally affixed to a given anchor. In this regard, it is well-known that following a given surgical procedure where a bone anchor is implemented to suspend or support a given suture, such attachment may unfortunately impart too much or too little tension or support. In such instances, the surgeon typically must perform yet another surgical procedure to make the necessary adjustments to the suture as attached to such bone anchor. As is well-known, to undergo yet a further surgical procedure causes substantial discomfort to the patient, substantially increases health care costs, and wastes health care resources.

Separate and apart from the aforementioned drawbacks is the failure of most prior art bone anchors to facilitate the attachment of ligaments, tendons (i.e., soft tissue) directly to bone. In this regard, most prior art bone anchors are inoperative to facilitate the direct interconnection between bone and soft tissue, and much less any ability to adjust the engagement (i.e., tension and positioning) of a tendon or a ligament to a specific target sight upon a bone.

In addition to the foregoing, there is yet a further substantial need in the art regarding the accurate placement of bone anchors such that the same are accurately and securely affixed into position. As discussed above, the methodology by which conventional bone anchors are secured into position is highly imprecise and, if incorrectly performed, leaves the surgeon with little alternative but to reattempt to accurately position such bone anchor. In this regard, there has not yet been available any methodology by which a precise target site of bone can be identified via a blind procedure, and much less any means to ensure that such properly identified target site secureably receives the bone anchor.

As such, there is a need in the art for a bone anchor and method of deploying the same that can be utilized as per conventional bone anchor mechanisms that further enables tension and/or support to be selectively imparted to a suture affixed thereto. There is additionally a need in the art for such a bone anchor that can be sized and adapted, and readily integrated into a wide variety of surgical applications, and may be further customized for use for a particular application such that an optimal degree of support and/or tension can be provided thereby. There is yet further a need in the art for such a bone anchor that can enable a physician or health care worker to post-operatively adjust in a non-surgical manner the tension and/or support imparted by a suture affixed to a bone anchor by enabling the bone anchor to selectively tighten or loosen the suture affixed thereto, and is further likewise capable of achieving such end via a relatively easy procedure that utilizes minimal manipulation. Still further, there is a need in the art for a bone anchor mechanism and attachment system that enables a tendon or ligament to be connected directly to bone that enables such interconnection between the ligament/tendon and bone to be selectively manipulated as may be desired for a given surgical procedure.

### BRIEF SUMMARY OF THE INVENTION

The present invention specifically addresses and alleviates the above-identified deficiencies in the art. In this regard, the present invention is directed to a bone anchor having means to selectively adjust the degree of tension imparted by a suture held thereby. According to a preferred embodiment, the bone anchor has an anchoring portion similar to those of the prior art. In this respect, such anchoring portion may comprise a threaded, self-tapping screw designed to be drilled directly into bone or posses a conical or generally arrowhead-shape configuration that is designed to be projected into the bone. Alternatively, such bone anchor may be formed to have a body portion operative to be seated completely across a given cross-section of bone and operative to define a channel through which a suture may be passed through a target site extending through a cross section of bone. In one preferred embodiment, such bone anchor will have a generally frusto-conical configuration operative to remain firmly seated across and within a target site of bone.

Formed upon the bone anchor is a suture receiving or attachment portion, which may comprise an eyelet, post, channel or other similar structure. With respect to the latter, however, the same is operatively coupled to an adjustment mechanism that can be selectively manipulated such that a suture tied and/or extending to such attachment portion can be selectively tightened or loosened to thus impart a desired degree of tension and/or support.

In a first embodiment, the attachment portion and adjustment mechanism comprise a conventional eyelet mounted upon a miniature ratchet system such that the eyelet can be selectively rotated in either a clockwise or counterclockwise fashion and thereafter be rigidly locked into position. Preferably, such ratchet system comprises a ratchet wheel mounted within an annular nesting structure, the latter having at least one, and preferably a plurality of pawls engageable with teeth formed radially about the ratchet wheel to thus cause the same to remain in a selectively fixed position. Such ratchet wheel may be operative to enable the eyelet to rotate clockwise and counterclockwise, or otherwise cause the eyelets to rotate in one direction.

In an alternative embodiment, such bone anchor comprises an anchor and an adjustment mechanism, the latter comprising a housing having a channel extending therethrough being operative to receive a suture. Formed within the housing is a brake mechanism operative to selectively impart a compressive frictional force against the suture extending through the housing such that the suture is caused to remain in fixed position. To that end, any break system, such as a plug, hook, or the like operative to selectively lock or compress the suture into position may be utilized. Preferably, such brake mechanism is biased downwardly via a spring that can be selectively manipulated such that the suture can be caused to selectively extend through the housing such that once a desired degree of tension and/support is imparted by the suture, the brake mechanism can be implemented to thus lock the suture line in fixed position.

In alternative configurations, the bone anchors of the present invention are provided with an anchoring portion that is operative to be seated completely across a target cross section of bone through which sutures and the like may be received. In this regard, the anchoring portion is operative to define a channel through a particular cross section of bone. Such bone anchor is further provided with an attachment portion that is operative to receive one or more sutures and selectively control the tension and/or degree of slack possessed thereby. According to preferred embodiments, such attachment portion may comprise a spool/ratchet mechanism whereby the sutures are caused to spool within the bone anchor to a degree such that a desired amount of tension and/or slack is possessed by the sutures held thereby. Alternatively, the attachment portion may include a ratchet wheel-type arrangement that likewise enables a desired degree of tension and/or slack to be possessed by the suture or sutures held thereby. Such mechanisms are further provided with means to remotely and/or atraumatically control the degree of tension/slack to thus enable post-operative adjustments to be made in a quick, accurate and painless manner.

In a further embodiment, there is provided a tendon/ligament fixation system operative to secure a tendon/ligament to a target site of bone. According to such embodiment, there is provided a bone anchor of the aforementioned embodiment operative to define a channel through a target site of bone through which a suture may be affixed. On one end of the channel formed on the side opposed from the bone anchor, there is provided a saddle member operative to be positioned about the opening defined by the channel. Such saddle is operative to define a socket into which a free end of a tendon/ligament may be seated. To facilitate such attachment, it is contemplated that a suture extending from the free end of the tendon/ligament will extend through the channel and operatively engage the bone anchor such that the free end of the ligament/tendon is forceably caused to seat within the saddle member formed on the opposed side of the channel extending through the bone. In a preferred embodiment such suture may comprise a self-anchoring suture line as disclosed in Applicant's co-pending application entitled Methods and Systems for Conjoining Tendons, Ligaments and the Like, the teachings of which are expressly incorporated herein by reference. By adjusting the tension at which the suture is held by the bone anchor formed on the opposed end of the channel to which the ligament/tendon is attached, such tension can be selectively adjusted as may be desired for a given procedure or post-operative adjustment.

In all embodiments, it is contemplated that the bone anchors and systems for securing ligaments/tendons to bone will be operative to be selectively adjusted in an atraumatic, quick and efficient manner which has not heretofore been available. It is expressly contemplated that such anchoring systems and methods will be particularly useful in cosmetic surgery procedures to enable various anatomical structures secured into position via the bone anchors of the present invention to be selectively positioned or re-positioned as may be desired to attain a more favorable patient outcome without the need to perform further surgery (or otherwise only perform minimally invasive surgery). In one specific application, it is contemplated that the anchoring mechanisms of the present invention may be operative to selectively adjust the positioning and tension at which an anatomical mass is held thereby, and in particular, an implant such as a breast implant, to thus enable the same to be optimally positioned post-surgically. Along these lines, the various embodiments of the present invention can be utilized to facilitate tendon distraction, a novel concept developed by Applicant which entails periodic stretching of the interconnected tendon/tendon or tendon/muscle over a set distance. As has not heretofor been available, such tendon distraction, by allowing for periodic stretching of the tendon/muscle complex, will allow for primary repair of tendons that otherwise will require tendon grafts of prosthesis to bridge long defects.

In addition to the foregoing, the present invention comprises methods for locating a target site at which to deploy a bone anchor utilizing a blind procedure that enables a surgeon to quickly and easily identify a target site upon a bone via an incision made through soft tissue and thereafter secure an anchor into position for use in further surgical manipulation that avoids a “trial and error” methodology of the prior art. Such method essentially comprises the step of introducing a channel extending from the target site to which a bone anchor is to be deployed. Through such channel, the target site is identified and thereafter a bore is formed thereat into which the bone anchor is seated. Advantageously, such procedure is exceptionally more accurate, fast, and atraumatic as compared to prior art bone anchor implant procedures, especially those that are performed blindly.

It is therefore an object of the present invention to provide a bone anchor operative to provide an optimal degree of tension or support to a suture affixed thereto, and in particular, provide means for selectively adjusting the degree of tension and/or support imparted by the suture affixed thereto.

Another object of the present invention is to provide a bone anchor having means for selectively adjusting the degree of tension possessed by a suture and/or the degree of support imparted thereby that can be manipulated post-operatively without requiring a further surgical procedure.

Another object of the present invention is to provide a system and method for attaching ligaments/tendons directly to bone to thus define a soft tissue/bone connection or interface that further can enable such conjoined structures to be selectively positioned or interconnected at a precise level of tension.

Another object of the present invention is to provide a bone anchor having means to selectively adjust the degree of tension of a suture affixed thereto or the degree of support imparted by a suture held thereby that is of simple construction, easy to manipulate, and provides the equivalent, if not greater, degree of support as per conventional bone anchors.

Another object of the present invention is to provide methods for deploying bone anchors that enable a bone anchor to be secured into position using a blind surgical procedure but yet further insures accurate placement of such bone anchor in a manner that is substantially faster and atraumatic than prior art blind bone anchor implantation procedures. Along these lines, the endoscope may be used as an addition to the procedure if needed without resorting to open incisions.

Still further objects of the present invention are to provide a bone anchor that can be readily utilized in a wide variety of surgical procedures and can be readily implemented utilizing existing bone anchor deployment technology, as well as novel methods for securing bone anchors and the like via a simple, expeditious and atraumatic manner that substantially minimizes medical complications and the uncertainty typically associated with securing bone anchors into position.

### BRIEF DESCRIPTION OF THE DRAWINGS

These as well as other features of the present invention will become more apparent upon reference to the drawings wherein:

FIG. 1 is a side perspective view of a prior art bone anchor shown imbedded within bone having a suture affixed thereto.

FIG. 2 is a side perspective view of a bone anchor constructed in accordance with a preferred embodiment of the present invention, such bone anchor being shown embedded in bone and operative to selectively impart a desired degree of tension to a suture attached thereto.

FIG. 3 is a top view taken along line 3-3 of FIG. 2.

FIG. 4 is a side perspective view of a bone anchor constructed in accordance with another preferred embodiment of the present invention and operative to selectively secure a suture extending therethrough in fixed position.

FIG. 5 is a top view of a bone anchor constructed in accordance with another preferred embodiment of the present invention.

FIG. 6 is a perspective view of the bone anchor depicted in FIG. 5, shown partially in phantom, defining a channel through which a suture may be extended.

FIG. 7 is a cross-sectional view of the bone anchor depicted in FIGS. 5 and 6 shown positioned through a cross section of bone with the top portion of the anchor shown in partially exploded view.

FIG. 8 is a perspective view of the body portion of a bone anchor constructed in accordance with yet a further embodiment of the present invention.

FIG. 9 is a perspective view of a tension adjustment mechanism positionable within the body portion depicted in FIG. 8.

FIG. 10 is a cross sectional view of a preferred embodiment of a bone anchor incorporating the housing and selective tension adjustment mechanism depicted in FIGS. 8 and 9, such bone anchor being shown positioned within a particular site of bone.

FIG. 11 is a perspective view of a bone anchor deployment system whereby a target site of bone is located for placement of a bone anchor.

FIG. 12 depicts the identification of a target site of bone within the system depicted in FIG. 11.

FIG. 13 depicts a procedure identifying and forming an orifice for positioning a bone anchor.

FIG. 14 depicts the formation of a hole upon a target site of bone utilizing a drill.

FIG. 15 depicts the deployment of a bone anchor into position upon a target site of bone.

FIG. 16 depicts the implantation of the bone anchor depicted in FIG. 15 with suture extending therethrough.

FIG. 17 depicts a perspective view of a further embodiment of the present invention operative to secure a ligament/tendon to a target site of bone.

FIG. 18 depicts the interconnection of a tendon to a bone utilizing the system of the present invention.

FIG. 19 is a cross-sectional view taken along a 19-19 of FIG. 18.

FIG. 20 is a perspective view of an anatomical mass/structure being supported by sutures, the latter being held into position via a bone anchor constructed in accordance with a preferred embodiment of the present invention.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood however, that the same or equivalent functions and sequences may be accomplished by different embodiments and that they are also intended to be encompassed within the scope of the invention.

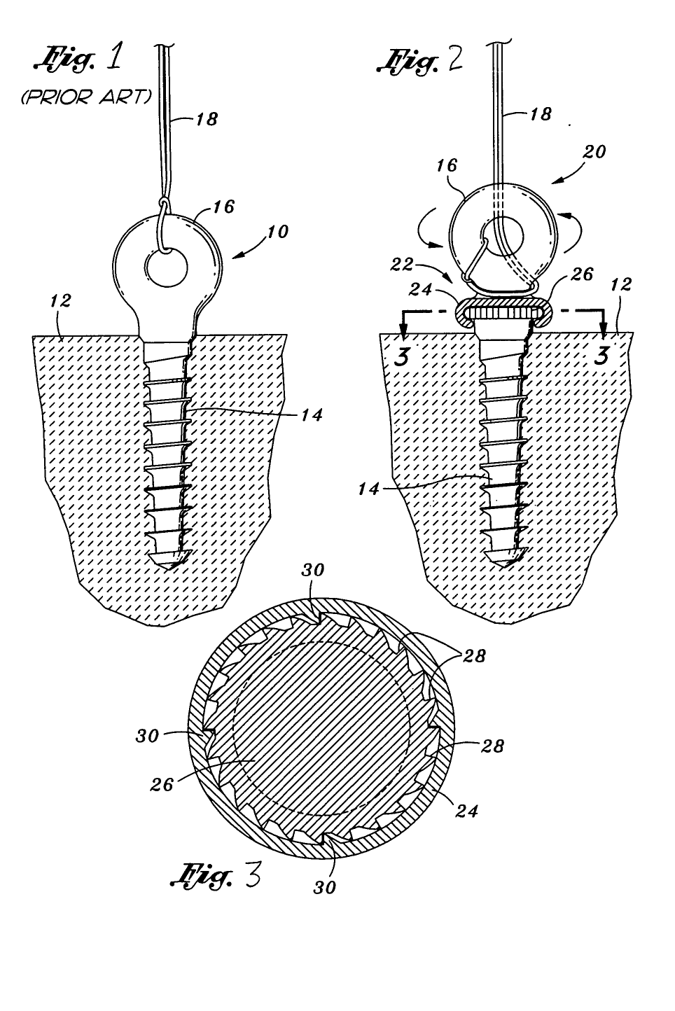

Referring now to the drawings, and initially to FIG. 1, there is shown a prior art bone screw 10 operative to remain securely embedded within a portion of bone 12 to thus form a secure, rigid structure to thus define an attachment point. Generally, such prior art bone screws comprise an anchoring portion, such as threaded portion 14 and an attachment portion, depicted as eyelet 16. With respect to the former, such anchoring portion 14, although depicted as a self-tapping bone screw, may also take other well-known configurations, such as generally arrowhead-like shaped or conical members operative to be projected into a target site of bone 12.

With respect to the attachment portion 16, although depicted as an eyelet, it is well-known that the same may take a variety of different configurations, and can include structures such as posts, hooks and the like. Such attachment portion 16 defines a structure to which a suture 18 may be tied, the latter being operative to suspend or support a secondary structure, such as a tissue mass, implant and the like. In this respect, such prior art bone anchors as depicted in FIG. 1, are well-known and extensively utilized in a variety of surgical procedures, and in particular orthopedic, gynecologic and plastic/reconstructive surgical procedures.

As is further well-known, however, such prior art bone anchors 10 as depicted in FIG. 1 frequently provide nothing more than an attachment point that remains static. As such, virtually all bone anchors are inoperative to selectively control the degree of tension and/or slack of the suture 18 affixed thereto. As such, once the bone anchor is affixed into position, surgeons must typically manipulate the suture 18 itself to thus control the necessary degree of tension and support imparted by the suture 18. As discussed in the Background, such approach is problematic insofar as the suture 18 frequently must be cut and/or retied until such time as an ideal tension and/or support is attained. Moreover, following the surgical procedure, it is often discovered that the degree of tension and/or support imparted by the suture 18 is less than optimal. However, given the inability to selectively adjust the tension and/or support imparted by the suture 18 post-operatively, the patient must typically undergo further surgical procedures.

To address such shortcomings, an improved bone anchor 20, depicted in FIG. 2, is provided that is operative to not only provide a rigid attachment point to which a suture 18 may be secured, but also provide means for selectively adjusting the tension possessed by suture 18 such that an optimal degree of tension and/or support is held by the suture 18. According to the embodiment shown, the bone anchor 20 possess an anchor portion 14, which may take any of a variety of well-known configurations in the art. As depicted, such anchor portion 14 comprises a self-tapping bone screw which is operative to be embedded within a portion of bone 20. As will be appreciated by those skilled in the art, such bone anchor may be installed utilizing conventional prior art mechanisms, such as bone-screw mechanisms. In an alternative embodiment not shown, such anchor portion 14 may take any of a variety of conical or arrowhead-shaped configurations that are operative to be projected into the bone via bone-anchor insertion devices.

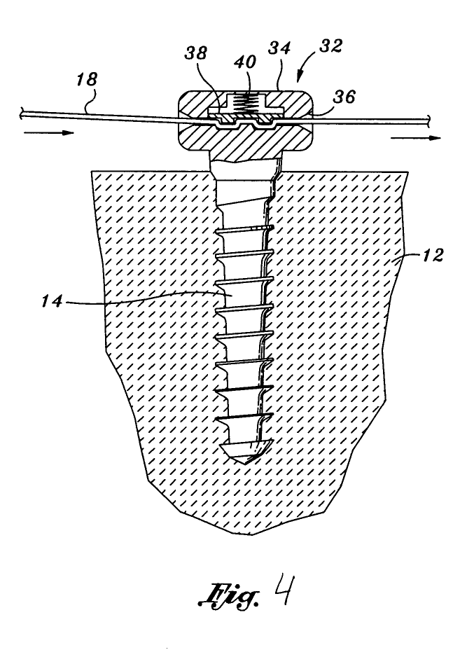

The bone anchor 20 further includes an attachment portion, such as eyelet 16, which again may take any of a variety of configurations known in the art operative to provide an attachment point for a suture 18, and can include hooks, posts, and the like. Unlike prior art bone anchors, however, such attachment portion is coupled with an adjustment mechanism 22 defined by ratchet wheel 26 and annular stop 24, the latter being more clearly seen in FIG. 3.

As illustrated, such arrangement between ratchet wheel 26 and annular stop 24 define a ratchet mechanism by which eyelet 16 is operative to rotate uni-directionally in the direction indicated by the letters in FIG. 2. To accomplish that end, ratchet wheel 26 is provided with a plurality of radially-arranged teeth 28 that are selectively engageable with at least one, and preferably a plurality of pawl members 30 formed on annular stop 24. As will be appreciated by those skilled in the art, by merely twisting eyelet 16 in the direction indicated by the letter “A” will thus cause suture 18 to spool about the eyelet 16 such that the degree of tension can be selectively increased. As will be further appreciated by those skilled in the art, by providing for such selective increase in the suture tension 18 advantageously enables the tension 18 to be gradually and incrementally adjusted and set without having to manipulate attachment of the suture 18 to the attachment portion, such as eyelet 16 in FIG. 1.

Although shown in its simplest form as a ratchet mechanism in FIGS. 2 and 3, it will be readily understood by those skilled in the art that a variety of mechanisms can be designed to selectively enable the attachment portion, in this case eyelet 16, to be selectively adjusted such that the same can impart a desired degree of tension to suture 18 affixed thereto. In this respect, it should be understood that adjustment mechanism 22 may be operatively configured to both increase and/or decrease the tension possessed by suture 18, and may be configured such that the attachment portion 16 is operative to move or rotate uni-directionally or bi-directionally to either increase tension, decrease tension, or both.

In addition to its functionality in providing a mechanism to selectively adjust the tension possessed by suture 18 affixed thereto, the bone anchor 20 of the present invention, and in particular the attachment portion 16 and adjustment mechanism 22 integrated therein, may be further designed and configured to be manipulated post-operatively. In this respect, to the extent suture 18 fails to possess the necessary tension and/or impart the desired degree of support to a given anatomical structure or implant, it is contemplated that the bone anchor 20 of the present invention may be non-surgically manipulated while in its seated position.

To accomplish that end, it is contemplated that attachment portion 16 may be configured to rotate via the application of external forces while embedded within the patient. In this regard, such adjustment may be made by the mere application of manual twisting. Alternatively, it is contemplated that such attachment portion 16 may be selectively and non-surgically manipulated by forming all or a portion of the attachment portion 16 from a magnetic substance and externally applying a magnetic force thereto such that the attachment portion 16 is caused to rotate or otherwise move in a manner sufficient to increase or decrease tension in suture 18 to thus attain a desired tension and/or support. As such, it should be readily understood by those skilled in the art that all variations by which such bone anchor 20 and in particular the adjustment mechanism 22 and attachment portion 16 thereof, can be manipulated in a non-surgical manner to adjust the tension in suture 18 affixed thereto should be deemed to be encompassed within the scope of the present invention.

Referring now to FIG. 4, there is shown the bone anchor of the present invention as constructed in accordance with a second preferred embodiment. As per the embodiment depicted in FIG. 2, the bone anchor includes an anchor portion 14 operative to remain securely embedded within bone 12. As discussed above, such anchor portion may take any of a variety of forms well-known in the art, including the self-tapping screw embodiment depicted in FIG. 4. Formed atop the anchor portion 14 is an attachment mechanism 32, which is operative to not only provide a secure point of attachment for a suture 18 but can also enable a surgeon to selectively control the degree of tension possessed by such suture.

To that end, the attachment mechanism 32 includes a housing 34, the latter having a channel 36 extending therethrough for receiving the suture 18. As illustrated, the suture 18 will preferably extend through such channel 36 to thus enable a surgeon to manipulate the tension held by such suture 18. Formed within housing 34 is a brake mechanism including brake member 38 operatively positioned to be compressed against suture 18 extending through channel 36. Such brake member 38 is preferably coupled to a downwardly-compressive spring 40, the latter being operative to compress brake member against suture 18 such that suture 18 is securely sandwiched into position within housing 34.

As will be appreciated by those skilled in the art, once the bone anchor is secured at a desired target site, the suture 18 will extend through channel 36 and manipulated (i.e., pulled) in the direction indicated by the letter “B” until such time as suture 18 optimally positions or supports a given structure. Once such optimal tension has been attained, the downwardly-compressive force imparted by spring 40 is operative to secure the suture 18 in its desired position. In this respect it is contemplated that spring 40 can be selectively deployed and operative to assume a compressed configuration, whereby channel 36 remains in an open state with the suture 18 free to extend therethrough, and an expansive configuration, as shown, whereby a compressive force is imparted to brake member 38 and operative to secure the suture 18 into position. In this respect, once the desired degree of tension has been attained, the suture 18 may be thereafter permanently secured into position.

In further refinements of the embodiment depicted in FIG. 4, it is contemplated that the same may be adapted for non-surgical, post-operative manipulation to thus enable the tension held by suture 18 to be selectively modified without having the patient to undergo further surgical procedures. To that end, it is contemplated that a magnetic element, which may be embedded within or encompass all of brake member 38, may be formed of a magnetic material such that application of a magnetic field is operative to cause brake member 38 to compress upwardly against spring 40, which in turn releases the suture 18 from its seated position within housing 34 and operative to adjust the tension held by such suture. As per the first embodiment, however, it is contemplated that a variety of alternative tension adjusting mechanisms will be readily-appreciated by those skilled in the art and that the scope of the present invention is deemed to encompass all such embodiments.

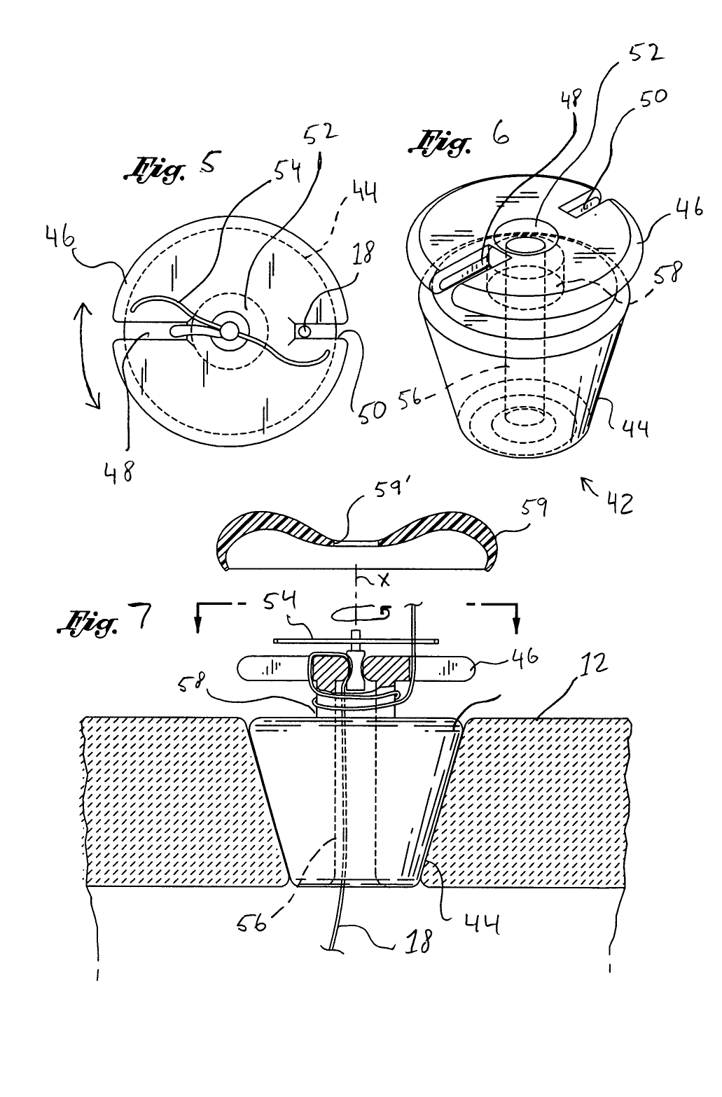

Referring now to FIGS. 5-7, there is shown a further embodiment 42 of a bone anchor constructed in accordance with the preferred embodiment of the present invention. Unlike the previous embodiments, the embodiment depicted is operative to define a channel extending completely through a section of bone through which sutures may be positioned and operatively adjusted to provide a desired degree of tension or support as depicted in FIG. 7. Such embodiment consists of a body portion or housing 44, which preferably has a generally frusto-conical shape operative to nest within a generally conically shaped bore made through bone 12. Disposed within such housing 44 is a channel 56 defining a passageway through which a suture 18 can extend. As will be appreciated by those skilled in the art, although depicted having a generally frusto-conical configuration, it will be recognized that such housing 44 may take any of a variety of well-known shapes and forms that are operative to define an anchor operative to be imbedded within bone.

Formed atop housing 44 is spool portion 46, the latter being operative to function as an anchor mechanism, as well as provide means for selectively adjusting the tension held by suture 18 coupled therewith. As illustrated, such spool portion 46 is preferably configured to have a generally annular shape with recesses 48,50 formed at diametrically opposed sides thereof. In use, the axial passageway 56, in combination with a respective one of the channels 48,50, will be operative to define a pathway through which the suture 18 will be positioned and selectively adjusted to the extent necessary.

To achieve that end, there is depicted in FIGS. 5 and 7 an adjustment mechanism 54 operative to selectively control the degree of tension placed upon suture 18. In this regard, such adjustment mechanism 54 preferably comprises at least one, and preferably a pair of diametrically-extending arms operative to rotate about an axis X, as depicted in FIG. 7. The arms of mechanism 54, once rotated about axis X, will be operative to increase or decrease the tension in suture 18. Advantageously, it is contemplated that such rotational mechanism 54 for adjusting tension may take any of a variety of configurations known in the art to thus enable the same to be adjusted remotely or via a minimally invasive procedure to the extent the same must necessarily be adjusted post operatively. Specifically, it is contemplated that mechanism 54 may be configured such that the same may be caused to rotate upon application of a magnetic field or adjusted via a percutaneous incision whereby a suitable device, such as a screw driver or other similar tool can impart the necessary rotational movement to mechanism 54 to thus enable the suture 18 to either tighten or loosen to a desired, selective degree. Advantageously, such mechanism not only provides for a means for adjusting the degree of tension supported by the suture 18, but further enables the same to be accomplished in extremely atraumatic and efficient manner that eliminates the need for such adjustment to be made by a further surgical procedure.

In order to protect mechanism 54, as well as spool portion 46 of the bone anchor 42, it is contemplated that an optional cap 59 may be provided that functions to selectively cover the top portion of such spool portion 46. In this regard, it is contemplated that such cap member 59 may be formed from a biocompatible material, such as silicone and the like, and thus serve to form a protective covering. Additionally, it is contemplated that such cover 59 may optionally include an aperture 59′ to the extent it is desired to leave an opening through which mechanism 54 can be accessed to the extent the same must necessarily be manipulated to control the degree of tension held by suture 18. It is likewise contemplated that such cap 59 may be operative to function as a stabilizing force such that mechanism 54 remains static unless otherwise manipulated by a treating physician.

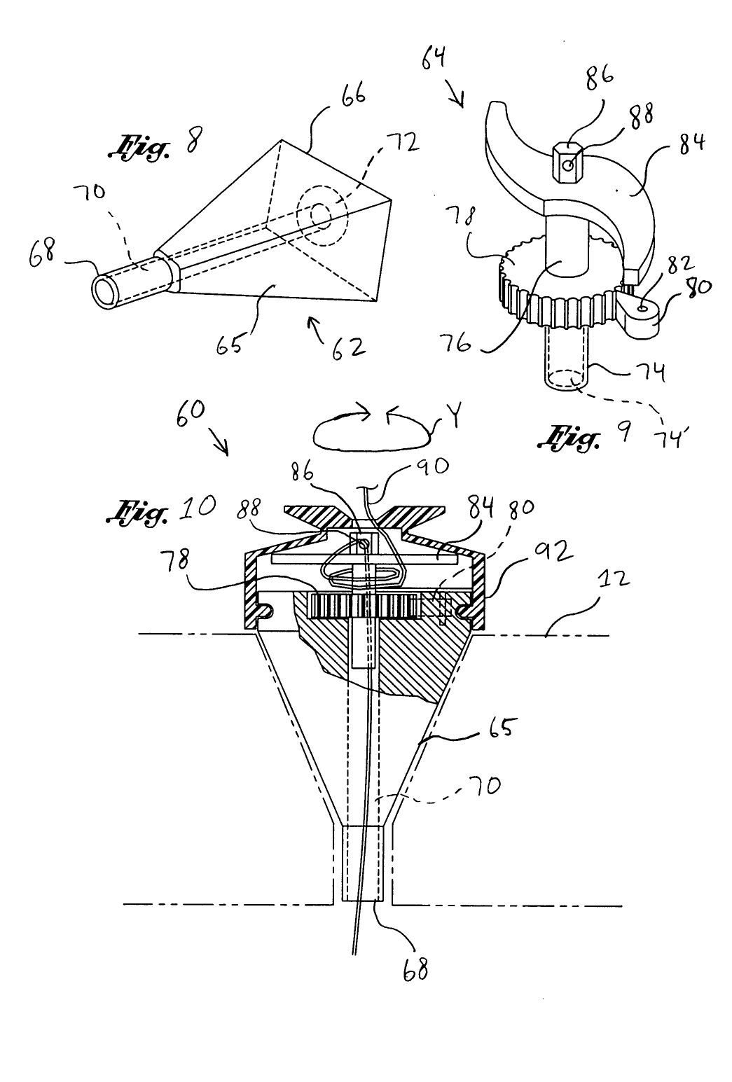

Referring now to FIGS. 8-10, there is shown yet another embodiment 60 of a bone anchor operative to secure and selectively control the degree of tension held within a suture line. Such embodiment 60 is comprised by the combination of a housing portion 62 depicted in FIG. 8, and a tension adjustment mechanism 64, depicted in FIG. 9. With respect to the former, the same includes a housing portion 65 defining an upper portion 66 through which the tension of the suture is selectively adjusted, and a distal portion 68 operative to define a channel through which the sutures are received through a bore made through bone 12. In this regard, such housing 62 will define a passageway 70 through which a suture 90 may extend, as depicted in FIG. 10.

The tension adjustment mechanism 64 depicted in FIG. 9, similar to the embodiment depicted in FIGS. 2 and 3, essentially relies on a ratchet-type system. As illustrated, such tension adjustment mechanism is provided with an elongate post 74 positionable within the housing 62 and defining a passageway 74′ through which the suture 90 will be received. A toothed wheel 78 is formed thereon and selectively engages with pawl or latch 80, the latter held in position via a pin mechanism received into orifice 82, as depicted in FIG. 10. A post 76 extending from the toothed wheel 78 extends upwardly upon which is formed adjustment mechanism 84, the latter defining diametrically extending portions operative to serve as a mechanism to twist to thus appropriately adjust the degree of tension held by suture 90. To that end, the adjustment mechanism 64 is provided with suture attachment housing 86, defining an eyelet 88 to which suture 90 may extend, as depicted in FIG. 10.

In use, the bone anchor 60 will be deposited in bone 12 and, as per the embodiment depicted in FIGS. 5-7, define a channel extending completely through a given cross section of bone. In this regard, it is contemplated that the exterior of housing 62 may take a variety of shapes and configurations known in the art which would enable the anchor 60 to remain firmly seated within an aperture formed within bone 12 to receive such housing. Once positioned therein, suture 90 will extend through passageway 70 and will be coupled to adjustment mechanism 64. With respect to the latter, suture 90 will extend through eyelet 88 and spool around post portion 76. Thereafter, via the selective rotation of adjustment member 84, suture 90 will be caused to rotate in the manner depicted by the letter Y to thus either selectively tighten or loosen suture 90. The ratchet mechanism provided by toothed wheel 78 and latch 80 will be operative to selectively set tension of suture 90 at a desired level.

To enhance protection of the adjustment mechanism 64, as well as maintain the degree of tension set thereby, it is contemplated that a protective housing 92, similar to 59, may be operatively positioned about the upper portion of housing 62 and adjustment mechanism 64. To that end, it is contemplated that protective housing 92 may be provided with a mechanism for interconnecting with housing 62 to thus enable the same to remain secured into position. Such covering 92 may further be provided with an aperture to facilitate the ability of the adjustment mechanism 64 to be selectively manipulated to the extent tension in the suture 90 must necessarily be adjusted. To that end, it is contemplated that all of the aforementioned techniques discussed above with respect to adjustment of tension in suture 90 may be incorporated in the embodiment 60 as shown. For example, it is contemplated that the adjustment mechanism 64 may be configured to be selectively controlled via the application of a magnetic field, or otherwise manipulated via minimally invasive surgical procedure, such as percutaneous adjustment.

Referring now to FIGS. 17-19, there is shown yet a further embodiment of the present invention, namely, a system 140 for attaching a ligament/tendon directly to bone. As illustrated, and which has not heretofore been available, tendon 146 may be anchored directly to bone 142 via the application of a bone anchor, formed in accordance with the aforementioned embodiments illustrated in FIGS. 5-10, in combination with a saddle member 144, the latter being operatively positioned over an aperture 152 through which a suture 150 may extend, as depicted in FIG. 17. Along these lines, it is contemplated that suture 150 may have preferably formed thereon a series of anchor elements 148 which thus enables the suture 150 to be firmly attached to tendon 146 and can enable the same to be selectively positioned, in this case within saddle 144 positioned over aperture 152. As illustrated, such anchoring mechanism takes the form of a plurality of pronged members formed along the length of suture 150. Although a variety of anchoring mechanisms may be known in the art which enable suture 150 to be connected to tendon 146, it is contemplated that the embodiment shown is indicative of a particularly preferred embodiment which takes the form of those systems disclosed in Applicant's co-pending patent application entitled System and Method for Conjoining Tendons, Ligaments and the Like, the teachings of which are expressly incorporated herein by reference. It will be understood, however, that alternative mechanisms may be utilized.

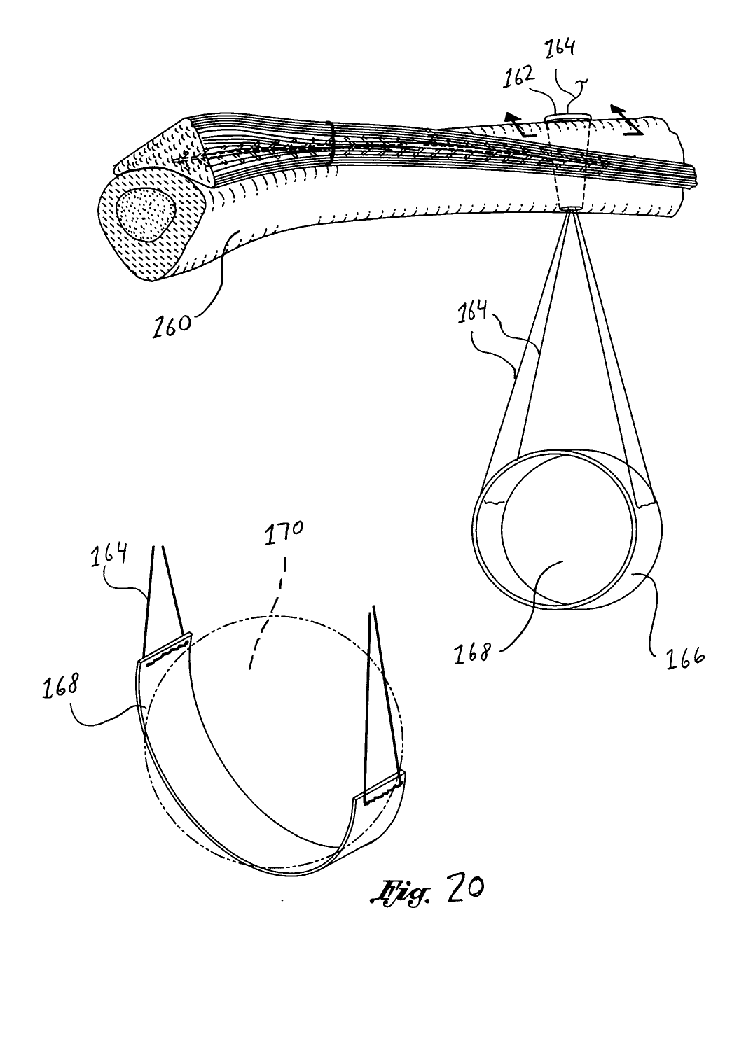

To secure the interconnection between tendon 146 and bone 142, it will be understood that a passageway will be formed within the bone 142, as depicted in FIG. 18. To the extent necessary, a larger bore may be formed, as depicted in the cross sectional view of FIG. 19 to thus enable the tendon 146 to be more fully received within the bone 142. Along these lines, it is contemplated that the size of the aperture formed within bone 142 may be formed as however may be desired to facilitate the attachment of a given tendon/ligament to the bone 142. As illustrated in FIG. 19, it is contemplated that a passageway 158 will be defined by housing 156 to thus accommodate the tendon 146 to be partially received therewithin.

Once such channel is formed, saddle member 144 will be positioned upon a respective end thereof and operative to define a seat or nest within which the free end of tendon 146 may be received. On the respective other opposed end of the channel formed through the bone 142 will be a bone anchor mechanism 154, which as discussed above, may take any of the aforementioned mentioned embodiments depicted in FIG. 5-10 to which suture 150 will be coupled. Along these lines, it is contemplated that the tension set in suture 150 may be selectively adjusted by the bone anchor 154 to thus enable the tendon 146 to be secured according to a desired tension and orientation. To that end, it is contemplated that bone anchor 154 may be selectively adjusted by any of the aforementioned mechanisms to thus ensure proper tension and secure attachment is maintained. Moreover, as has not heretofor been available, such novel approach for repairing tendons can provide means for a new medical concept, namely, tendon distraction, which entails periodic stretching of the tendon and muscle connected thereto over a specific distance. As will be appreciated by those skilled in the art, by providing means for such periodic stretching of the tendon/muscle complex will allow for primary repair of tendons that otherwise will require tendon grafts or prosthesis to bridge long defects.

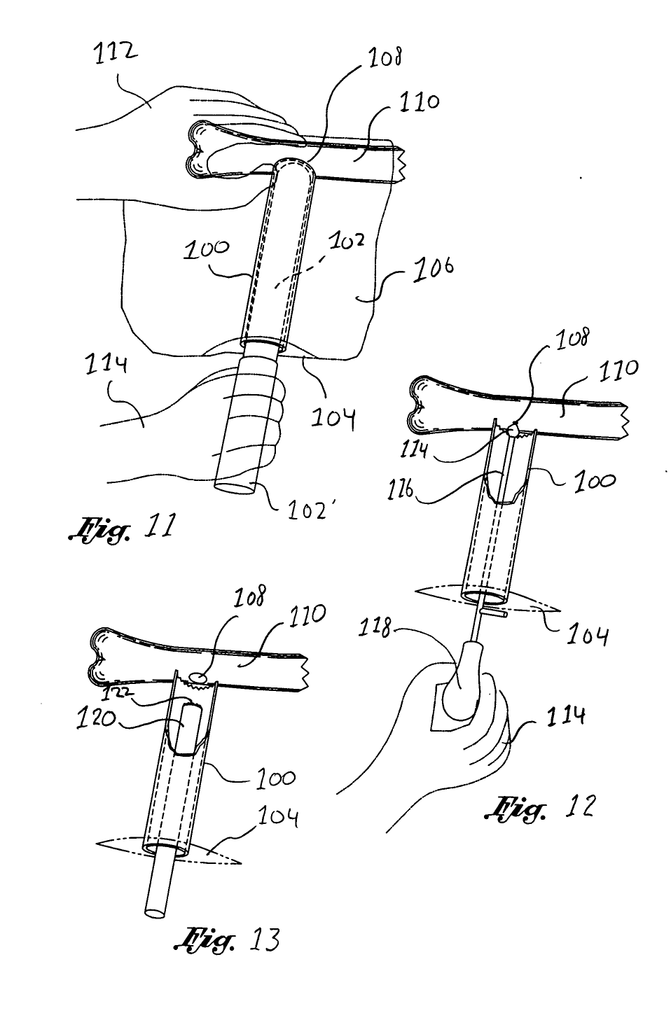

In addition to the various aforementioned embodiments for securing sutures and the like to bones, as well as the advantageous means by which the same can be selectively manipulated, the present invention further discloses novel methods for quickly and accurately identifying target sites upon a particular bone to where a bone anchor may be deployed, as well as the rapid and accurate fixation of such anchor upon such target site. Advantageously, such methods enable a bone anchor to be deployed blindly but in a manner that is also substantially less traumatic, expeditious and accurate compared to conventional bone deployment techniques (although it will be readily appreciated that such bone anchor devices of the present invention may be deployed endoscopically or by other means well-known in the art). Referring now to FIGS. 11-16, there is sequentially illustrated the procedure by which the bone anchors of the present invention, or any other prior art bone anchors for that matter, may be quickly, easily and accurately secured into position at a target site upon a bone.

Referring initially to FIG. 11, such deployment procedure involves the initial step of positioning a cylindrical tool 100 through an incision 104 made into surrounding tissue 106. Through such incision 104, the cylindrical tool is advanced upwardly to a target site 108 of bone 110. As will be recognized by those skilled in the art, the specific bone in question may be readily identified via a basic understanding of anatomy and confirmed via external palpitation or manual manipulation, as accomplished by hand 112. In order to guide cylindrical portion 112 into position, it is contemplated that a guiding member 102 with handle portion 102′ held by the other hand of the physician 114 will be utilized to guide and advance the cylindrical portion up against the target site 108 of bone 110. Once the cylindrical portion is caused to remain in abutment with the target site 108 of bone 110, as confirmed via a perceptible contact between the cylinder 100 and bone 110, the deployment member 102 is removed with the cylinder 100 remaining in position in abutment against bone at target site 108, as depicted in FIG. 12.

While in such orientation, the surgeon is provided with a cylindrical channel through which a bone anchor deployment procedure may be performed. To that end, it is contemplated that any of a variety of tools may be deployed through cylinder 100 to form a bore into which an anchor may be positioned. As depicted in FIG. 12, it is contemplated that a simple marking mechanism 116 having a marking tip 114 may be deployed to provide a marking at the target site 108 as may be necessary to insure accurate placement of the bone anchor via the deployment of a bone anchor under direct vision. Alternatively, it is contemplated that such procedure may simply involve directly screwing a bone anchor into position, as per the embodiments depicted in FIGS. 1-4 to the extent it is not necessary to form a passageway completely through the bone 110.

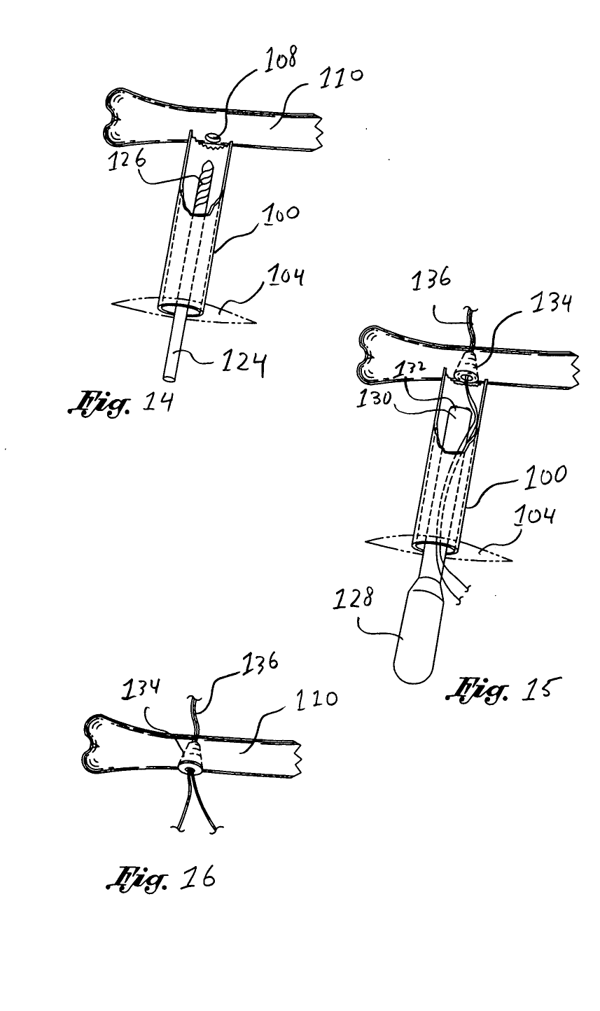

Alternatively, as depicted in FIGS. 13-14, once the cylinder 110 has been properly positioned about target site 108, a suitable drilling mechanism, such as a boring instrument 120 having a jagged edged distal end 122 depicted in FIG. 13 or drill 124 having drill bit 126 depicted in FIG. 14, may be utilized to form the appropriate aperture into which the bone anchor will be seated. In this regard, it is contemplated that whatever appropriate instrument is utilized to form the passageway through the bone 110, the same will do so precisely or near precisely at the selected target site 108.

Once the appropriate channel has been formed, the bone anchor will thus be positioned therewithin, such as 134 depicted in FIG. 15. According to such illustration, it is contemplated that the bone anchor 134 will be seated within the aperture formed within the bone via a bone anchor deployment mechanism 128, the latter having an elongate portion 130 with distal end 132, the latter being operative to selectively position the bone anchor 134 at the target site. It is further expressly contemplated that in order to facilitate the positioning and manipulation of sutures through such bone anchor 134, such sutures 136 may be concurrently deployed with the bone anchor 134 such that the same are maintained in a surgically operable position, as shown.

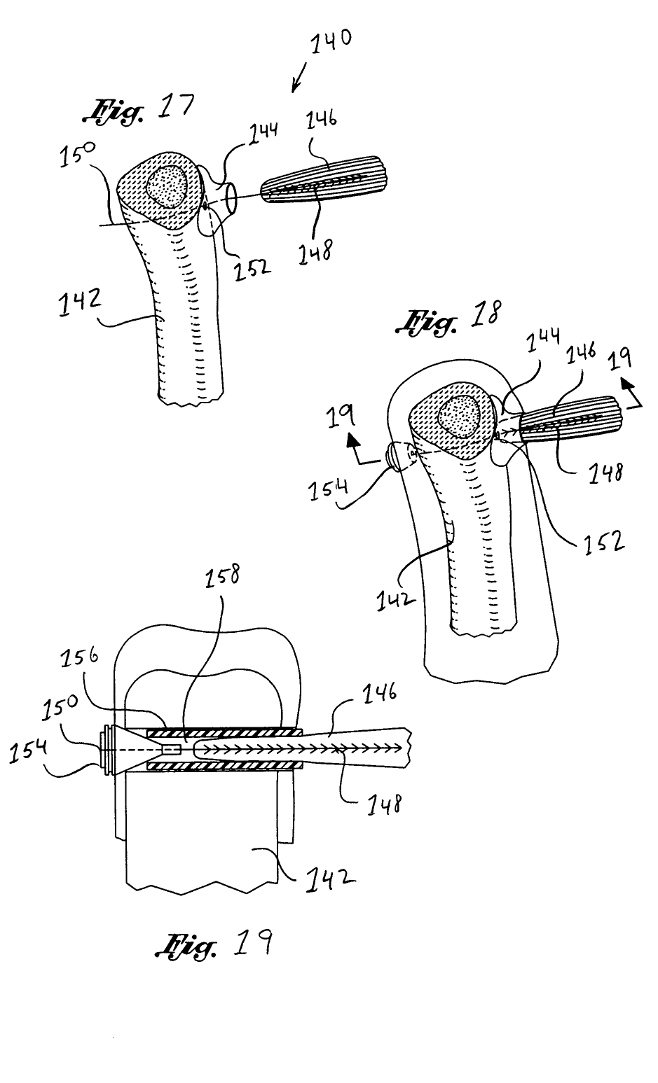

Once the bone anchor 134 is secured into position, the deployment mechanism 128 and cylinder 100 defining the passageway within which the same is deployed may be removed through suture 104 to thus enable the bone 110 with bone anchor 134 with sutures 136 extending therthrough to remain in place, as depicted in FIG. 16. Once so positioned, the anchors thus will serve as an attachment point to which the suture 136 can be selectively manipulated and secured into position according to a desired tension such that a given anatomical mass or structure supported thereby is maintained in optimal orientation.

FIG. 20 is illustrative of such application whereby a bone anchor 162, which may take any of the aforementioned variety, is coupled with sutures 164 secured thereto and extending therefrom. In this regard, bone anchor 162 defines a passageway through the bone 160, the latter of which will serve as a support structure to which an anatomical mass can be supported. To that end, it is contemplated that sutures 164 will be coupled to a graft, sling, or some other type of anatomical support structure operative to define a cavity 168 within which may be securably positioned an anatomical mass or structure such as implant 170 shown in phantom. Along these lines, it is expressly contemplated that the anchor mechanisms of the present invention will be particularly well suited for use in cosmetic surgery, and in particular in breast augmentation which may provide a novel means by which to support an implant, such as 170 such that the same is not only maintained in an optimal position, but is also maintained such that the degree of support provided to the implant via sutures 164 and sling 168 affixed thereto can be selectively adjusted to insure proper placement and orientation to attain as favorable a patient outcome as possible.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

1. A bone anchor operative to selectively control the tension of a suture held thereby comprising:
a. an anchor portion operative to be seated within a bone mass;
b. an attachment portion coupled to said anchor portion, said attachment portion defining an attachment point to which said suture is affixed to said bone anchor; and
c. an adjustment mechanism operatively coupled to said attachment portion, said adjustment mechanism being operative to selectively adjust said attachment portion such that the tension of said suture affixed thereto is correspondingly altered.

2. The bone anchor of claim 1 wherein said attachment portion is selected from the group consisting of an eyelet, hook, and post.

3. The bone anchor of claim 1 wherein said anchor portion comprises a self-tapping threaded screw.

4. The bone anchor of. Claim 1 wherein said anchor portion comprises an anchor element configured to be projected within said bone.

5. The bone anchor of claim 1 wherein said adjustment mechanism comprises a ratchet mechanism disposed intermediate said attachment portion and said anchor portion.

6. The bone anchor of claim 5 wherein said ratchet mechanism is operative to permit uni-directional rotational movement of said attachment member.

7. The bone anchor of claim 5 wherein said ratchet mechanism is operative to permit bi-directional rotational movement of said attachment member.

8. A bone anchor operative to selectively control the tension of a suture held thereby comprising:
a. an anchor portion operative to be seated within a bone mass; and
b. an attachment mechanism coupled to said anchor portion, said attachment mechanism being operative to receive and selectively engage said suture such that said suture is held in selective position and possess a selective tension relative said bone anchor.

9. The bone anchor of claim 8 wherein said bone anchor further comprises:
a brake mechanism disposed within said attachment portion of said bone anchor, said brake mechanism being operative to compressively hold said suture received in said attachment mechanism.

10. The bone anchor of claim 9 wherein said brake system comprises the combination of a brake member and a spring element, said brake member and said spring element being operatively interconnected such that said spring element biases said brake member against said suture disposed within said attachment member.

11. The bone anchor of claim 7 wherein said attachment member is formed from a magnetic material.

12. The bone anchor of claim 10 wherein said brake member comprises a magnetic material.

13. A bone anchor operative to selectively control the tension of a suture held thereby comprising:
a. an anchor portion operative to be seated across a bone mass, said anchor portion defining a channel extending through said bone mass;
b. an attachment portion defining an attachment point to which said suture is affixed to said bone anchor; and
c. an adjustment mechanism operatively coupled to said anchor portion and said attachment portion, said adjustment mechanism being operative to selectively adjust said attachment portion such that the tension of said suture affixed thereto is correspondingly altered.

14. The bone anchor of claim 13 wherein said adjustment mechanism comprises a ratchet mechanism disposed within said channel formed within said bone mass.

15. The bone anchor of claim 14 further comprising a protective covering positionable about said adjustment mechanism.

16. The bone anchor of claim 14 wherein said bone anchor further includes a spool portion operative to capture and hold a suture segment coiled thereabout.

17. A system for selectively attaching a tendon or ligament to a bone comprising:
a. an anchor portion operative to be seated across a bone mass, said anchor portion defining a channel extending through said bone mass;
b. an attachment portion defining an attachment point to which said suture is affixed to said bone anchor; and
c. an adjustment mechanism operatively coupled to said anchor portion and said attachment portion, said adjustment mechanism being operative to selectively adjust said attachment portion such that the tension of said suture affixed thereto is correspondingly altered;
d. a saddle member operatively positionable upon a respective open end of said channel formed through said bone mass, said saddle member being operative to receive and hold a free end of a tendon or a ligament thereupon, said saddle member having a bore formed therethrough in fluid communication with said channel formed through said bone mass, said bore forming a passageway through which a suture coupled to said tendon may extend to said bone anchor.

18. A method of securing a bone anchor to bone comprising the steps:
a. forming a channel extending from said target site of bone and through a section of soft tissue such that said channel extends externally from the body of said patient;
b. identifying a site through said channel formed in step (a) to where said bone anchor will be positioned;
c. forming a bore within said bone at said site identified in step (b); and
d. depositing said bone anchor within said bore formed in step (c).

19. The method of claim 18 wherein in step (a), said channel is formed by forming an incision upon said section of soft tissue and advancing a cylindrical sleeve through said soft tissue to said target site of said bone.

20. The method of claim 18 wherein in step (c), said bore is formed partially through said target site of bone.

21. The method of claim 18 wherein in step (c), said bore is formed completely through said target site of bone such that a channel extending through said target site of bone is formed.

22. The method of claim 21 wherein in step (d), said bone anchor is positioned across said channel formed through said target site of bone.
